(19) United States
(12) Patent Application Publication
(10) Pub. No.: US 2015/0157273 A1
(43) Pub. Date: Jun. 11, 2015

(54) HEART FAILURE EVENT PREDICTION USING CLASSIFIER FUSION

(71) Applicant: Cardiac Pacemakers, Inc., St. Paul, MN (US)

(72) Inventors: Qi An, Blaine, MN (US); Guy R. Hotson, Baltimore, MD (US); Pramodsingh Hirasingh Thakur, Woodbury, MN (US); Yi Zhang, Plymouth, MN (US)

(21) Appl. No.: 14/540,834

(22) Filed: Nov. 13, 2014

Related U.S. Application Data
(60) Provisional application No. 61/912,568, filed on Dec. 6, 2013.

(51) Int. Cl. A61B 5/00; A61B 7/04; A61B 5/11; A61B 5/0205

(52) U.S. Cl. CPC A61B 5/7264; A61B 5/0205; A61B 7/04; A61B 5/1118; A61B 5/1121; A61B 5/7275; A61B 5/7282; A61B 5/024

(57) ABSTRACT

Systems and methods for detecting a heart failure (HF) event indicative of worsening of HF, or for identifying patient at elevated risk of developing future HF event, are described. The system and methods can detect an HF event or predict HF risk using a multitude of fusion algorithms or classifiers, each employing one or more physiologic sensor signals. A system can comprise two or more partial predictor circuits each can adaptively generate a dynamic computational model (DCM). Each partial predictor circuit can determine a partial risk index indicating a likelihood of the patient developing a precursor physiologic event indicative or correlative of a future HF event. The system can include a prediction fusion circuit that can combine the partial risk indices and generate a composite risk indicator for detecting or predicting a likelihood of the patient developing a future HF event.

### CLAIM OF PRIORITY

This application claims the benefit of priority under 35 U.S.C. §119(e) of U.S. Provisional Patent Application Ser. No. 61/912,568, filed on Dec. 6, 2013, which is herein incorporated by reference in its entirety.

### TECHNICAL FIELD

This document relates generally to medical devices, and more particularly, to systems, devices and methods for detecting and monitoring worsening of heart failure.

### BACKGROUND

Congestive heart failure (CHF) is a major health problem and affects over five million people in the United States alone. CHF is the loss of pumping power of the heart, resulting in the inability to deliver enough blood to meet the demands of peripheral tissues. CHF patients typically have enlarged heart with weakened cardiac muscles, resulting in reduced contractility and poor cardiac output of blood.

CHF is usually a chronic condition, but can occur suddenly. It can affect the left heart, right heart or both sides of the heart. If CHF affects the left ventricle, signals that control the left ventricular contraction can be delayed, and the left and right ventricles do not contract simultaneously. Non-simultaneous contractions of the left and right ventricles further decrease the pumping efficiency of the heart.

### OVERVIEW

Frequent monitoring of CHF patients and timely detection of events indicative of heart failure (HF) decompensation status can help prevent worsening of HF in CHF patients, hence reducing cost associated with HF hospitalization. Identification of patient at an elevated risk of developing future HF events such as worsening of HF can help ensure timely treatment, thereby improving the prognosis and patient outcome. On the other hand, identifying and safely managing the patients at low risk of future HF events can avoid unnecessary medical intervention and reduce healthcare cost.

Ambulatory medical devices can be used for monitoring HF patient and detecting HF decompensation events. Examples of such ambulatory medical devices can include implantable medical devices (IMD), subcutaneous medical devices, wearable medical devices or other external medical devices. The ambulatory or implantable medical devices can include physiologic sensors which can be configured to sense electrical activity and mechanical function of the heart, or physical or physiological variables associated with the signs or symptoms associated with a new or worsening of an existing disease, such as pulmonary edema, pulmonary condition exacerbation, asthma and pneumonia, myocardial infarction, dilated cardiomyopathy, ischemic cardiomyopathy, systolic HF, diastolic HF, valvular disease, renal disease, chronic obstructive pulmonary disease, peripheral vascular disease, cerebrovascular disease, hepatic disease, diabetes, asthma, anemia, depression, pulmonary hypertension, sleep disordered breathing, or hyperlipidemia, among others.

The medical device can optionally deliver therapy such as electrical stimulation pulses to a target area, such as to restore or improve the cardiac function or neural function. Some of these devices can provide diagnostic features, such as using transthoracic impedance or other sensor signals. For example, fluid accumulation in the lungs can decrease the transthoracic impedance due to the lower resistivity of the fluid than air in the lungs. Fluid accumulation in the lungs can also irritate the pulmonary system and leads to decrease in tidal volume and increase in respiratory rate. In another example, heart sounds can be useful indications of proper or improper functioning of a patient's heart. Heart sounds are associated with mechanical vibrations from activity of a patient's heart and the flow of blood through the heart. Heart sounds recur with each cardiac cycle, and according to the activity associated with the vibration, heart sounds can be separated and classified into various components including S1, S2, S3, and S4 heart sounds.

Because the worsening of HF, such as a HF decompensation event, can be a complex process resulting in systematic changes in patient's physiology, a single physiologic sensor may not always provide desired performance in timely and accurately detecting or predicting the worsening of HF. Some ambulatory medical devices can include multiple physiologic sensors working cooperatively to detect or predict the worsening of HF. For example, an ambulatory medical device can include a fusion center that combines the responses of multiple physiologic sensors to create a decision about the worsening of HF.

Although such a sensor fusion may to some extent overcome the limitation of a single physiologic sensor in detecting or predicting worsening of HF, challenges still remain. For example, a sensor fusion algorithm, or a classifier, is usually designed such that it can detect a type of change in the signatures of the physiologic sensors that are correlative of a particular physiologic manifestation or symptom of worsening of HF. However, worsening of HF status may not consistently be associated with such changes in the sensor signatures; rather, there can be a wide variety of pathophysiologic manifestations across HF patients. Even within a patient, the pathophysiologic manifestation can vary significantly from one HF decompensation event to another. For example, within a patient or across a patient population, HF decompensation events can have a presentation of wet-versus-dry profile which indicates the presence and level of congestion, or a presentation of cold-versus-warm profile which indicates adequacy of the systematic blood circulation. Worsening of HF may also be clinically manifested as a peripheral congestion or a central congestion. Furthermore, worsening of HF can be associated with substantially elevated left-ventricular filling pressure in some patients but not in others. Additionally, worsening of HF can be triggered by different types of comorbidities co-existing in the patients, such as atrial arrhythmia, COPD, pneumonia, hypertension, diabetes and renal dysfunction. Because of high level of intra-patient and inter-patient variations in the symptoms and clinical manifestations of worsening of HF, a particular fusion algorithm, even employing multiple physiologic sensors, may not be robust and reliable enough to accurately and timely detect the progression of the HF in a wide range of patients. The present inventors have recognized that there remains a considerable need of systems and methods that can detect target physiologic events indicative of worsening of HF or identify CHF patients with elevated risk of developing future events of worsening of HF with improved accuracy and reliability, particularly in an ambulatory setting.

Various embodiments described herein can help improve detection of an HF event indicative of worsening of HF, or improve process of identifying patients at elevated risk of developing future target physiologic event, such as a future HF events. The present inventors have recognized that by using a multitude of fusion algorithms or classifiers each employing signals acquired from a plurality of physiologic sensors, a classifier fusion scheme that pools the outputs from the multitude of fusion algorithms can further improve the reliability and the robustness of detecting or predicting the worsening of HF. For example, a system can comprise a physiologic signal receiver circuit, two or more partial predictor circuits, and a prediction fusion circuit. The physiologic signal receiver circuit can receive at least one physiologic signal obtained from a patient. Each of the two or more partial predictor circuits can generate one or more candidate signal features from the at least one physiologic signal. The partial predictor circuit can include a dynamic computational model (DCM) circuit configured to adaptively generate a DCM. Using the one or more candidate signal features and the DCM, the partial predictor circuit can calculate a partial risk index which indicates a likelihood of the patient developing a precursor physiologic event indicative or correlative of a future heart failure (HF). The prediction fusion circuit can be coupled to the two or more partial predictor circuits, and can generate a composite risk indicator using the partial risk indices produced by the two or more partial predictor circuits, where the composite risk indicator can be indicative of a likelihood of the patient developing the future target physiologic event.

A system can comprise a dynamic computational model unit and an ambulatory medical device (AMD) communicatively coupled to the dynamic computational model (DCM) unit. The DCM unit can include a memory circuit configured to receive and store physiologic data, and a model update circuit configured to adaptively generate two or more dynamic computational models using the stored physiologic data. The AMD can include a physiologic signal receiver circuit configured to receive at least one physiologic signal obtained from a patient and a receive circuit that can receive from the dynamic computational model unit the two or more dynamic computational models. The AMD can include two or more partial predictor circuits that can generate one or more candidate signal features from the at least one physiologic signal, and calculate a partial risk index using the one or more candidate signal features and the two or more dynamic computational models. The partial risk index can indicate a likelihood of the patient developing a precursor physiologic event indicative or correlative of a future target physiologic event, such as a future heart failure (HF) event. The AMD can also include a prediction fusion circuit that can generate a composite risk indicator using the partial risk indices produced by two or more partial predictor circuits, where the composite risk indicator can be indicative of a likelihood of the patient developing the future target physiologic event.

A method can include receiving at least one at least one physiologic signal obtained from a patient and generating one or more candidate signal features using the at least one physiologic. The method can include adaptively generating at least first and second dynamic computational models, and calculating a first partial risk index using first signal features and the first dynamic computational model and calculating a second partial risk index using second signal features and the second dynamic computational model. The first and second signal features can be respectively selected from the one or more candidate signal features. The first and second partial risk indices can respectively indicate likelihood of the patient developing first and second precursor physiologic events. Both the first and second precursor physiologic events can be indicative or correlative of a future target physiologic event, such as a future heart failure (HF) event. The method includes generating a composite risk indicator using one or both of the first and second partial risk indices. The composite risk indicator can be indicative of a likelihood of the patient developing the future target physiologic event.

This Overview is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof, each of which are not to be taken in a limiting sense. The scope of the present invention is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates an example of a cardiac rhythm management (CRM) system and portions of the environment in which the CRM system operates.

FIG. 2 illustrates an example of a classifier-fusion based HF event prediction circuit.

FIG. 3 illustrates another example of a classifier-fusion based HF event prediction circuit.

FIG. 4 illustrates an example of an ensemble of predictors and the prediction fusion of the output from the ensemble of predictors.

FIG. 5 illustrates an example of a method for predicting a patient's risk of developing an event indicative of worsening of HF.

FIG. 6 illustrates an example of a method for generating a computational model used for predicting a physiologic event or a pathophysiologic manifestation of an impending worsening of HF.

### DETAILED DESCRIPTION

Disclosed herein are systems, devices, and methods for detecting an event indicative of worsening of HF such as an HF decompensation event, or for identifying patients with elevated risk of developing future events related to worsening of HF. The HF event detection or risk stratification can be performed using the physiologic signals such as sensed from one or more physiologic sensors associated with an ambulatory medical device such as an implantable cardiac device. The physiologic signals can be selectively used by two or more partial predictors each of which is configured to calculate a partial risk index indicating a likelihood of the patient developing a precursor physiologic event indicative or correlative of worsening of HF. A classifier fusion can then be used to combine the partial risk indices and generate a detection decision of worsening of HF, or to predict the risk of future HF event, thereby allowing immediate medical attention to the patient.

FIG. 1 illustrates an example of a Cardiac Rhythm Management (CRM) system 100 and portions of an environment in which the CRM system 100 can operate. The CRM system 100 can include an ambulatory medical device, such as an implantable medical device (IMD) 110 that can be electrically coupled to a heart 105 such as through one or more leads 108A-C, and an external system 120 that can communicate with the IMD 110 such as via a communication link 103. The IMD 110 may include an implantable cardiac device such as a pacemaker, an implantable cardioverter-defibrillator (ICD), or a cardiac resynchronization therapy defibrillator (CRT-D). The IMD 110 can include one or more monitoring or therapeutic devices such as a subcutaneously implanted device, a wearable external device, a neural stimulator, a drug delivery device, a biological therapy device, a diagnostic only device, or one or more other ambulatory medical devices. The IMD 110 may be coupled to, or may be substituted by a monitoring medical device such as a bedside or other external monitor.

As illustrated in FIG. 1, the IMD 110 can include a hermetically sealed can 112 that can house an electronic circuit that can sense a physiological signal in the heart 105 and can deliver one or more therapeutic electrical pulses to a target region, such as in the heart, such as through one or more leads 108A-C. The CRM system 100 can include only one lead such as 108B, or can include two leads such as 108A and 108B.

The lead 108A can include a proximal end that can be configured to be connected to IMD 110 and a distal end that can be configured to be placed at a target location such as in the right atrium (RA) 131 of the heart 105. The lead 108A can have a first pacing-sensing electrode 141 that can be located at or near its distal end, and a second pacing-sensing electrode 142 that can be located at or near the electrode 141. The electrodes 141 and 142 can be electrically connected to the IMD 110 such as via separate conductors in the lead 108A, such as to allow for sensing of the right atrial activity and optional delivery of atrial pacing pulses. The lead 108B can be a defibrillation lead that can include a proximal end that can be connected to IMD 110 and a distal end that can be placed at a target location such as in the right ventricle (RV) 132 of heart 105. The lead 108B can have a first pacing-sensing electrode 152 that can be located at distal end, a second pacing-sensing electrode 153 that can be located near the electrode 152, a first defibrillation coil electrode 154 that can be located near the electrode 153, and a second defibrillation coil electrode 155 that can be located at a distance from the distal end such as for superior vena cava (SVC) placement. The electrodes 152 through 155 can be electrically connected to the IMD 110 such as via separate conductors in the lead 108B. The electrodes 152 and 153 can allow for sensing of a ventricular electrogram and can optionally allow delivery of one or more ventricular pacing pulses, and electrodes 154 and 155 can allow for delivery of one or more ventricular cardioversion/defibrillation pulses. In an example, the lead 108B can include only three electrodes 152, 154 and 155. The electrodes 152 and 154 can be used for sensing or delivery of one or more ventricular pacing pulses, and the electrodes 154 and 155 can be used for delivery of one or more ventricular cardioversion or defibrillation pulses. The lead 108C can include a proximal end that can be connected to the IMD 110 and a distal end that can be configured to be placed at a target location such as in a left ventricle (LV) 134 of the heart 105. The lead 108C may be implanted through the coronary sinus 133 and may be placed in a coronary vein over the LV such as to allow for delivery of one or more pacing pulses to the LV. The lead 108C can include an electrode 161 that can be located at a distal end of the lead 108C and another electrode 162 that can be located near the electrode 161. The lead 108C can include one or more electrodes in addition to the electrodes 161 and 162 along the body of the lead 108C. The electrodes 161 and 162, and any additional electrodes on the lead 108C, can be electrically connected to the IMD 110 such as via separate conductors in the lead 108C such as to allow for sensing of the LV electrogram and optionally allow delivery of one or more resynchronization pacing pulses from the LV.

The IMD 110 can include an electronic circuit that can sense a physiological signal. The physiological signal can include an electrogram or a signal representing mechanical function of the heart 105. The hermetically sealed can 112 may function as an electrode such as for sensing or pulse delivery. For example, an electrode from one or more of the leads 108A-C may be used together with the can 112 such as for unipolar sensing of an electrogram or for delivering one or more pacing pulses. A defibrillation electrode from the lead 108B may be used together with the can 112 such as for delivering one or more cardioversion/defibrillation pulses. In an example, the IMD 110 can sense impedance such as between electrodes located on one or more of the leads 108A-C or the can 112. The IMD 110 can be configured to inject current between a pair of electrodes, sense the resultant voltage between the same or different pair of electrodes, and determine impedance using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing. In an example, the IMD 110 can be configured to inject current between an electrode on the RV lead 108B and the can housing 112, and to sense the resultant voltage between the same electrodes or between a different electrode on the RV lead 108B and the can housing 112. A physiologic signal can be sensed from one or more physiological sensors that can be integrated within the IMD 110. The IMD 110 can also be configured to sense a physiological signal from one or more external physiologic sensors or one or more external electrodes that can be coupled to the IMD 110. Examples of the physiological signal can include one or more of heart rate, heart rate variability, arrhythmia information, intrathoracic impedance, intracardiac impedance, arterial pressure, pulmonary artery pressure, left atrial pressure, RV pressure, LV coronary pressure, coronary blood temperature, blood oxygen saturation, one or more heart sounds, physical activity or exertion level, physiologic response to activity, posture, respiration, body weight, or body temperature.

The arrangement and functions of these leads and electrodes are described above by way of example and not by way of limitation. Depending on the need of the patient and the capability of the implantable device, other arrangements and uses of these leads and electrodes are possible.

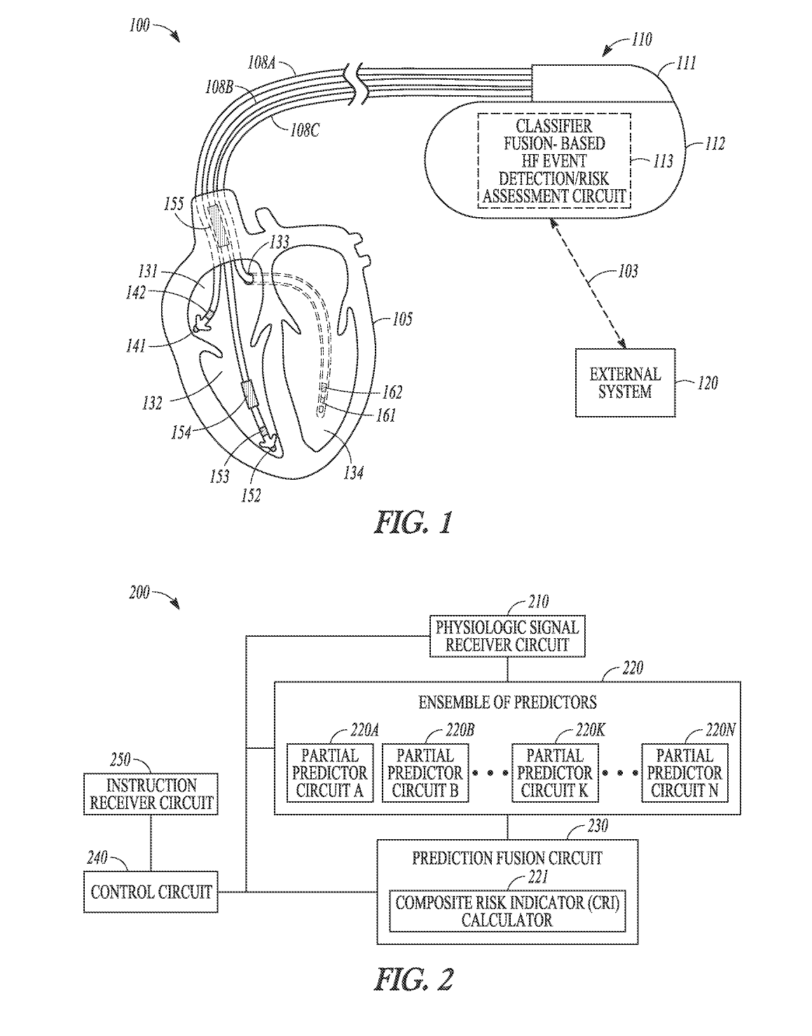

As illustrated, the CRM system 100 can include a classifier-fusion based HF event detection/risk assessment circuit 113. The classifier-fusion based HF event detection or risk assessment circuit 113 can receive at least one physiologic signal obtained from a patient. The physiologic signals can be bio-electrical or mechanical signals that are indicative or correlative of worsening of HF. For example, the physiologic signals can include electrograms sensed using ambulatory physiologic sensors deployed on or within the patient and communicated with the IMD 110, such as electrodes on one or more of the leads 108A-C and the can 112. The physiologic signal can also include signals sensed by one or more ambulatory sensors, including blood pressure signals, heart sound signals, bio-impedance signals, respiration signals, posture, activity, heart rate or activity signals, or physiological response to activity (PRA) signals, among others. The classifier-fusion based HF event detection or risk assessment circuit 113 can include an ensemble of partial predictors. Each predictor can be capable of adaptively generating a dynamic computational model (DCM) that operates on one or more physiologic signals or signal features obtained from the physiologic signals, and calculating a partial risk index indicating a likelihood of the patient developing a precursor physiologic event indicative or correlative of a future heart failure (HF) event. Examples of such precursor physiologic events can include a pulmonary or a cardiac event, or a central or a peripheral congestion event, among others. The classifier-fusion based HF event detection or risk assessment circuit 113 can generate a composite risk indicator using the partial risk indices, where the CRI can be indicative of the likelihood of the patient developing a future event of worsening of HF. Examples of the classifier-fusion based HF event detection or risk assessment circuit 113 are described below, such as with reference to FIGS. 2-4.

The external system 120 can allow for programming of the IMD 110 and can receive information about one or more signals acquired by IMD 110, such as can be received via a communication link 103. The external system 120 can include a local external IMD programmer. The external system 120 can include a remote patient management system that can monitor patient status or send commands to the IMD 110 such as to program diagnostic functions or to adjust one or more therapies such as from a remote location.

The communication link 103 can include one or more of an inductive telemetry link, a radio-frequency telemetry link, or a telecommunication link, such as an internet connection. The communication link 103 can provide for data transmission between the IMD 110 and the external system 120. The transmitted data can include, for example, real-time physiological data acquired by the IMD 110, physiological data acquired by and stored in the IMD 110, therapy history data or data indicating IMD operational status stored in the IMD 110, one or more programming instructions to the IMD 110 such as to configure the IMD 110 to perform one or more actions that can include physiological data acquisition such as using programmably specifiable sensing electrodes and configuration, device self-diagnostic test, or delivery of one or more therapies.

The classifier-fusion based HF event detection or risk assessment circuit 113 may be implemented at the external system 120, which can be configured to perform HF risk stratification such as using data extracted from the IMD 110 or data stored in a memory within the external system 120. Portions of the classifier-fusion based HF event detection or risk assessment circuit 113 may be distributed between the IMD 110 and the external system 120. In an example, the DCMs used by each of the ensemble of partial predictors can be implemented in the external system 120.

Portions of the IMD 110 or the external system 120 can be implemented using hardware, software, or any combination of hardware and software. Portions of the IMD 110 or the external system 120 may be implemented using an application-specific circuit that can be constructed or configured to perform one or more particular functions, or can be implemented using a general-purpose circuit that can be programmed or otherwise configured to perform one or more particular functions. Such a general-purpose circuit can include a microprocessor or a portion thereof, a microcontroller or a portion thereof, or a programmable logic circuit, or a portion thereof. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. While described with reference to the IMD 110, the CRM system 100 could include a subcutaneous medical device (e.g., subcutaneous ICD, subcutaneous diagnostic device), wearable medical devices (e.g., patch based sensing device), or other external medical devices.

FIG. 2 illustrates an example of a classifier-fusion based HF event prediction circuit 200, which can be an embodiment of the classifier-fusion based HF event detection or risk assessment circuit 113. Additionally or alternatively, the classifier-fusion based HF event prediction circuit 200 can be implemented in an external system such as a local or remote patient monitor or patient management system such as the external system 120, which is configured for providing the patient's diagnostic information to an end-user.

The classifier-fusion based HF event prediction circuit 200 can include one or more of a physiologic signal receiver circuit 210, an ensemble of predictors 220, a prediction fusion circuit 230, a controller circuit 240, and an instruction receiver circuit 250. The physiologic signal receiver circuit 210 can be configured to receive one or more physiological signals that can be indicative or correlative of progression of a patient's HF status, such as worsening of HF. The physiologic signals can be sensed using one or more physiologic sensors implanted within or attached to the patient. Examples of such a physiological signal can include one or more electrograms sensed from the electrodes on one or more of the leads 108A-C or the can 112, heart rate, heart rate variability, arrhythmia information, intrathoracic impedance, intracardiac impedance, arterial pressure, pulmonary artery pressure, left atrial pressure, RV pressure, LV coronary pressure, coronary blood temperature, blood oxygen saturation, one or more heart sounds, physiologic response to activity, apnea hypopnea index, one or more respiration signals such as a respiration rate signal or a tidal volume signal. The physiologic signals can also include one or more of brain natriuretic peptide (BNP), blood panel, sodium and potassium levels, glucose level and other biomarkers and bio-chemical markers. The physiologic signals can be acquired from a patient and stored in a storage device such as an electronic medical record (EMR) system. The physiologic signal receiver circuit 210 can be coupled to the storage device and retrieve from the storage device one or more patient historical physiologic signals in response to a command signal. The command signal can be issued by a system user (e.g., a health-care professional) such as via an input device coupled to the instruction receiver 250, or generated automatically by the system in response to a specified event. The physiologic signal receiver circuit 210 can include one or more sub-circuits that can perform signal conditioning or pre-processing, including signal amplification, digitization, or filtering, on the one or more physiological signals.

The ensemble of predictors 220 can include two or more partial predictor circuits such as partial predictor circuit A 220A, predictor circuit B 220B, predictor circuit K 220K, . . . , predictor circuit N 220N. Each partial predictor can be configured to adaptively generate a dynamic computational model (DCM) that operates on the at least one physiologic signal provided by the physiologic signal receiver circuit 210, or signal features generated using the physiologic signals. A DCM can include one or a combination of various model types including a rule-based model, a decision tree model, a regression model, a neural network model, a random forest, a voting model, a fuzzy logic model, a support vector machine model or any mathematical model that operates on time-series data. A DCM can be directed to a specified patient group that has certain types of comorbidities or certain triggering events. A DCM can include several elements that constitute the configuration of the model. For example, a decision tree model can include such elements as nodes, paths, and levels.

The type and configuration of the DCMs can be initialized independently for the partial predictors. Each DCM can be independently updated such as using a feedback mechanism. Adaptation of a DCM can include update of the structure or complexity of the model when the model output meets a specified criterion. In an example, portions of the physiologic data can be randomly selected to feed into the DCM. The output of the DCM, such as a categorical decision or classification of a precursor physiologic event indicative or correlative of worsening of HF, can be compared to an adjudication of the precursor physiologic event. The adjudication can be provided by a health-care professional or an end-user. The DCM can be updated when the comparison between the model output and the adjudication meets a specified criterion.

Each partial predictor circuit can calculate a partial risk index using the DCM and the one or more physiologic signals. The partial risk index can characterize a particular aspect of the patient's pathophysiology correlated to the worsening of HF. For example, the partial risk index can be a probability value indicating a likelihood of the patient developing a future precursor physiologic event indicative or correlative of worsening of HF, such as a pulmonary or a cardiac event, a central or a peripheral congestion event, or other events.

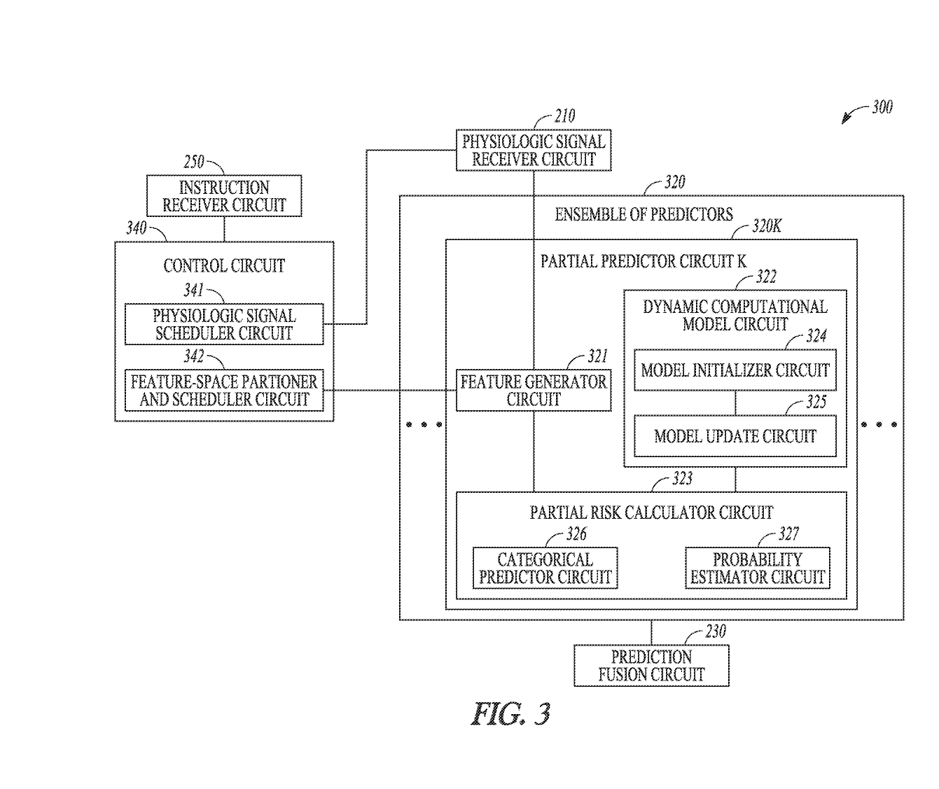

The partial predictor circuits 220A-220N can differ from each other by the respective DCM, or by the physiologic signals used for calculating the partial risk index. The partial risk indices produced by the partial predictor circuits 220A-220N can respectively correspond to different types of precursor physiologic events. Examples of the partial predictor circuits are described below, such as with reference to FIG. 3.

The prediction fusion circuit 230, coupled to the two or more partial predictor circuits 220A-220N, can include a composite risk indicator (CRI) calculator 231 configured to calculate a CRI using the partial risk indices produced by the two or more partial predictor circuits 220A-220N. The CRI can be indicative of a likelihood of the patient developing a future event of worsening of HF, such as an HF decompensation event. The CRI can be a classification decision taken from two or more classes indicating various levels of likelihood of having an impending event of worsening of HF. For example, when the two or more partial predictor circuits 220A-220N each produces a partial risk index in a form of a categorical decision indicating presence or absence of a particular pathophysiologic manifestation, the prediction fusion circuit 230 can compute the CRI using decision fusion of the categorical decisions produced by the two or more partial predictor circuits 220A-220N. Examples of the decision fusion method can include majority voting, X-out-of-Y voting, or weighted voting, among others. The CRI can also be a continuous quality such as a probability value indicating a likelihood of occurrence of an impending event of worsening of HF. For example, when the two or more partial predictor circuits 220A-220N each produces a partial risk index in a form of a probability value (such as between 0 and 1) indicating the confidence level of a prediction of an impending pathophysiologic manifestation, the prediction fusion circuit 230 can be configured to compute the CRI using probability fusion of the probability values produced by the two or more partial predictor circuits 220A-220N. Examples of the probability fusion method can include linear or a non-linear combination such as average or weighted summation, or parametric or non-parametric methods such as a decision tree, a neural networks, a Bayesian network, among other machine learning methods.

The prediction fusion circuit 230 can generate a report to inform, warn, or alert a system end-user an elevated risk of a patient developing a future HF event. The report can include the CRI with corresponding timeframe within which the risk is predicted. The report can also include recommended actions such as confirmative testing, diagnosis, or therapy options. The report can be presented in one or more media formats including, for example, a textual or graphical message, a sound, an image, or a combination thereof. In an example, the report can be presented to the user via an interactive user interface on the instruction receiver circuit 250. The prediction fusion circuit 230 can also generate and present to the end-user, such as via the external device 120 or the instruction receiver circuit 250, one or more of a report including information about adaptation of the DCM and the partial risk index computed within each partial predictor circuit.

The controller circuit 240 can control the operations of the physiologic signal receiver circuit 210, the ensemble of predictors 220, the prediction fusion circuit 230, and the data flow and instructions between these components. The controller circuit 240 can receive external programming input from the instruction receiver circuit 250. The instruction receiver circuit 250 can include a user interface configured to present programming options to the user and receive user's programming input. In an example, at least a portion of the instruction receiver circuit 250, such as the user interface, can be implemented in the external system 120.

The controller circuit 240 can control one or more of sensing physiologic signals and generating signal features from the physiologic signals, initializing and updating adaptively the DCMs, calculate a partial risk indices, or performing prediction fusion such as by generating the composite risk indicator. The control circuit 240 can also include sub-circuits that select, for each of the two or more partial predictor circuits 220A-220N, one or more physiologic signals or a portion of the signal features generated from the physiologic signals to be used for calculating the partial risk index. Examples of the control circuit 240 are described below, such as with reference to FIG. 3.

FIG. 3 illustrates an example of a classifier-fusion based HF event prediction circuit 300, which can be an embodiment of the classifier-fusion based HF event detection or risk assessment circuit 113, or an embodiment of the classifier-fusion based HF event prediction circuit 200. The classifier-fusion based HF event prediction circuit 300 can include one or more of a physiologic signal receiver circuit 210, an ensemble of predictors 320, a prediction fusion circuit 230, a controller circuit 340, and an instruction receiver circuit 250.

As discussed in FIG. 2, the physiologic signal receiver circuit 210 can be configured to receive one or more physiological signals that can be indicative of worsening of HF status. Examples of the physiologic signals can include electrocardiogram, intracardiac electrograms, heart rate signal, heart rate variability signal, arrhythmia information, cardiac impedance signal, thoracic impedance signal, arterial pressure signal, pulmonary artery pressure signal, left atrial pressure, RV pressure signal, LV coronary pressure signal, coronary blood temperature signal, blood oxygen saturation signal, one or more heart sounds, physiologic response to activity, apnea hypopnea index, one or more respiration signals such as a respiration rate signal or a tidal volume signal. The physiologic signals can also include one or more of brain natriuretic peptide (BNP), blood panel, sodium and potassium levels, glucose level and other biomarkers and bio-chemical markers. The physiologic signal receiver circuit 210 can receive the physiological signals using one or more communicatively coupled ambulatory physiologic sensors associated with the patient. Alternatively or additionally, the physiologic signal receiver circuit 210 can receive the physiologic signals from a storage device such as an electronic medical record (EMR) system that stores one or more physiologic signals.

The ensemble of predictors 320, which can be an embodiment of the ensemble of predictors 220, can include two or more partial predictor circuits, each of which can be an embodiment of the respective partial predictor circuits 220A-220N. For example, an exemplary partial predictor circuit K 320k can be an embodiment of the partial predictor circuit K 220K. The partial predictor circuit K 320k can be configured to calculate a partial risk index that characterizes a particular aspect of the patient's pathophysiology related to the worsening of HF. The partial risk index can be a categorical decision or a continuous quality representing a probability value indicative of a likelihood of the patient later developing a precursor physiologic event such as a pulmonary or a cardiac event, a central or a peripheral congestion event, or other events indicative of worsening of HF.

As an example of the two or more partial predictor circuits in the ensemble of predictors 320, the partial predictor circuit K 320k can include one or more of a feature generator circuit 321, a dynamic computational model (DCM) circuit 322, and a partial risk calculator circuit 323. The partial predictor circuits in the ensemble of predictors 320 can differ from each other by the respective DCM, by the physiologic signals provided to the respective generation circuit, or by signal features used by respective partial risk calculator circuit. The partial risk indices produced by the partial predictor circuits can respectively correspond to different types of precursor physiologic events.

The feature generator circuit 321, coupled to the controller circuit 340, can be configured to generate one or more candidate signal features from the at least one physiologic signal such as provided by the physiologic signal receiver circuit 210. The controller circuit 340, which can be an embodiment of the controller circuit 240, can include a physiologic signal scheduler circuit 341 and a feature-space partitioner and scheduler circuit 342. The physiologic signal scheduler circuit 341 can control the physiologic signals to be used by each individual partial predictor circuit. In an example, the physiologic signal scheduler circuit 341 can select different physiologic signals for the various partial predictor circuits. In another example, the physiologic signal scheduler circuit 341 can selected the physiologic signals for the partial predictors such than two different partial predictors circuits may share one or more physiologic signals but has at least one different physiologic signal used for generating the respective partial risk index. By using non-identical physiologic signals, various partial predictor circuits can characterize non-identical manifestations of the patient's pathophysiology indicative or correlative of the worsening of HF.

As illustrated in FIG. 3, the feature generator circuit 321 can select, from a plurality of physiologic signals received by the physiologic signal receiver circuit 210, one or more physiologic signals for the partial predictor circuit K 320K. For example, the physiologic signal scheduler circuit 341 can select for the partial predictor circuit K 320k one or more thoracic impedance signals such as sensed by two or more electrodes disposed on one or more leads 108A-C or the can 112, including an impedance vector sensed between an RA electrode 141 or 142 and the can 112 (Z_{RA-Can}), between an RV electrode 152, 153 or 154 and a can 112 (Z_{RV-Can}), or between an LV electrode 161 or 162 and the can 112 (Z_{RV-Can}). The thoracic impedance signal can also include an impedance vector where the voltage sensing electrodes are the currently injection electrodes are orthogonal to each other, such as selected from RA, RV, or LV electrodes (Z_{RA-RV-LV}). The physiologic signal scheduler circuit 341 can select for another partial predictor circuit M (not shown) an acoustic or vibrational heart sound (HS) signal such as sensed by an ambulatory accelerometer, an ambulatory microphone, or other heart sound sensors either external to or implanted inside patient body. The physiologic signal scheduler circuit 341 can select other different physiologic signal to the partial predictor circuit K and the partial predictor circuit M, such as a respiration signal, a physiologic response to activity (PRA) signal, or a posture signal, among others.

The feature generator circuit 321 can generate from the selected physiologic signals one or more candidate signal features. Examples of signal features can include: signal mean, median, or other central tendency measures; a histogram of the signal intensity; one or more signal trends over time; one or more signal morphological descriptors; one or more signal change or rate of change features; one or more signal change or rate of change features, or signal power spectral density at a specified frequency range. The signals features can include components corresponding to physiologic activities. For example, the electrocardiogram or electrogram features can include P wave, R wave, T wave, QRS complex, or other components representing depolarization, hyperpolarization, repolarization, or other electrophysiological properties of the myocardium. The heart sound features can include timing, amplitude, or morphologic characteristics of one or more of S1, S2, S3, or S4 heart sounds. The thoracic impedance features can include maximum, minimum, mean, variance, rate of change, or other statistical or morphological features. The respiration signal features can include respiration rate, respiration depth, tidal volume, or other descriptors.

The feature-space partitioner and scheduler circuit 342 can be configured to select different signal features for the various partial predictor circuits in the ensemble of predictors 320. By using different signal features, various partial predictor circuits can characterize non-identical pathophysiologic manifestations indicative or correlative of the worsening of HF. In an example, the feature-space partitioner and scheduler circuit 342 can select for the partial predictor circuit K 320k signal features that are sensitive or specific to a pulmonary event such as a pulmonary edema, asthma and pneumonia, chronic obstructive pulmonary disease; and select for another partial predictor circuit M (not shown) signal features that are sensitive or specific to a cardiac event such as a atrial or ventricular arrhythmia, myocardial infarction event, coronary artery disease, heart attack event, percentage of pacing or bi-ventricular pacing received by the patient, amount of pacing relative to amount of sensing of cardiac activations. Examples of the signal features used for detecting or predicting a pulmonary event can include one or a combination of daily average transthoracic impedance, tidal volume, respiration rate, or apnea-hypopnea index among others. Examples of the signal features used for detecting or predicting a cardiac event can include one or any a combination of intrathoracic impedance, pulmonary arterial pressure, activity level, posture, S1 heart sound strength, S3 heart sound strength, systolic timing interval, or pre-ejection and ejection time, among others. Additionally, the feature-space partitioner and scheduler circuit 342 can also select for the partial predictor circuit K 320k signal features that are sensitive or specific to certain disease conditions including a renal disease, a diabetic condition, or hypertension. Examples of the signal features used for detecting or predicting a renal event can include creatinine, body urea nitrogen (BUN), BUN/creatinine ratio and glomerular filtration rate (GFR). Examples of the signal features used for detecting or predicting a diabetic condition include glucose level. Examples of the signal features used for detecting or predicting a hypertensive condition include blood pressure measurement.

In an example, the feature-space partitioner and scheduler circuit 342 can select for the partial predictor circuit K 320k signal features that are that are sensitive or specific to a central congestion, and select for the partial predictor circuit M (not shown) signal features that are that are sensitive or specific to a peripheral congestion. Examples of the signal features used for detecting or predicting central congestion can include one or any combination of transthoracic impedance, respiratory rate, tidal volume, left-ventricular filling pressure, or pulmonary capillary wedge pressure. Examples of the signal features used for detecting or predicting peripheral congestion can include one or any combination of respiratory rate during exertion, activity, body weight, S1 heart sound amplitude, systolic time interval, or blood pressure.

In another example, the feature-space partitioner and scheduler circuit 342 can select for the various partial predictor circuits signal features that can be used to characterize certain wet-versus-dry profile or cold-versus-warm status in a HF patient. The wet-versus-dry profile can suggest presence and level of congestion, while the cold-versus-warm status can suggest hemodynamic status of a patient, particularly the adequacy of the systematic blood circulation. A target profile, such as a warm-and-dry presentation, can indicate a well-managed HF status where the patient is free from severe congestion and maintains adequate peripheral perfusion. A worsening of HF status may be accompanied by diversified presentations such as cold-and-wet, cold-and-dry, or warm-and-wet presentations. The feature-space partitioner and scheduler circuit 342 can select for the partial predictor circuit K 320k signal features that are sensitive or specific to the warm-and-wet profile, and select for the partial predictor circuit M (not shown) signal features that are sensitive or specific to a cold-and-dry profile of the worsening of HF. Examples of the signal features used for detecting or predicting the warm-and-wet profile or the cold-and-dry profile can include one or any combination of transthoracic impedance, respiratory rate, tidal volume, S1 heart sound amplitude, systolic time interval, weight, left-ventricular filling pressure, or pulmonary capillary wedge pressure.

The feature-space partitioner and scheduler circuit 342 can select signal features for the partial predictor circuits when the patient has one or more specified types of comorbidities or underlying diseases. The partial predictor circuits can therefore characterize pathophysiologic manifestations indicative or correlative of the worsening of HF for a specified subgroup of patients with such comorbidities or underlying diseases. For example, the feature-space partitioner and scheduler circuit 342 can select for the partial predictor circuit K 320k signal features acquired from patients with renal disease, and select for the partial predictor circuit M (not shown) signal features acquired from patients with hypotension. The signal features provided to the partial predictor circuit K 320k can be sensitive or specific to cardiac events, thus allowing the partial predictor circuit K 320k to detect or predict a cardiac event indicative or correlative of worsening of HF in patients with renal disease. The signal features provided to the partial predictor circuit M can be sensitive or specific to pulmonary events, thus allowing the partial predictor circuit M to detect or predict a pulmonary event indicative or correlative of worsening of HF in patients with hypertension condition.

In some examples, either or both of the physiologic signal schedule circuit 341 and the feature-space partitioner and scheduler circuit 342 can be coupled to the instruction receiver 250, such as via an input device, and receive a command or programming instructions from an end-user such as a health-care professional. For example, the end-user can program the ensemble of predictors such that the partial predictor circuit K, among other partial predictors, can receive specified physiologic signals, generate signal features using the received physiologic signals, and to predict a pulmonary edema event preempting a worsening of HF, or a central congestion or a warm-and-wet profile that characterize an impending worsening of HF.

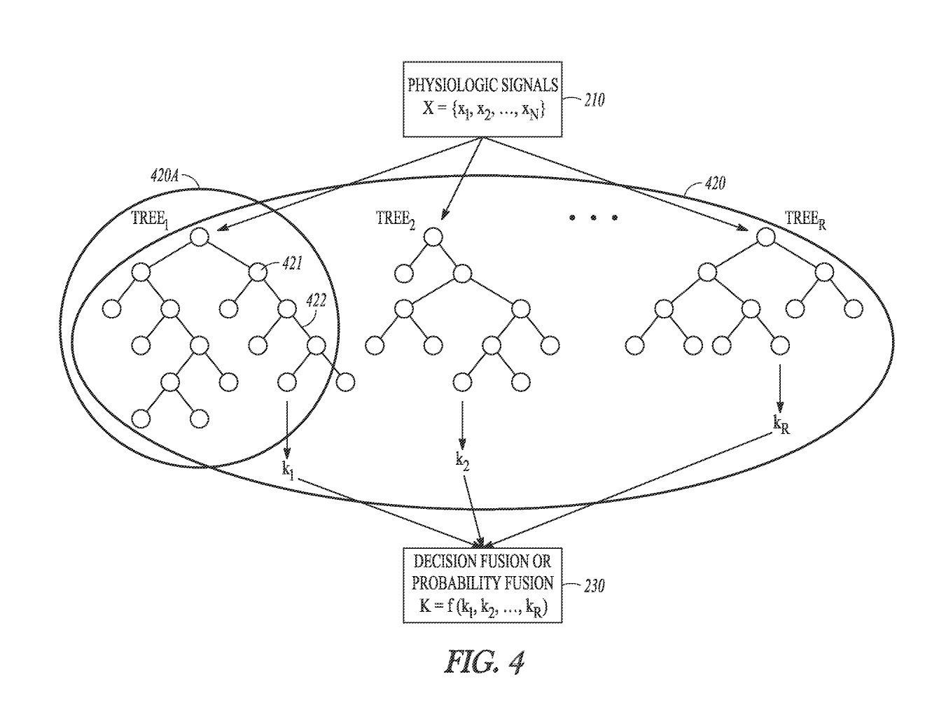

The DCM circuit 322 can be configured to adaptively generate a DCM that operates on the selected physiologic signals or the signal features generated by the feature generator circuit 321. A computational model can be a specified set of processor-executable instructions stored in a memory. The DCM circuit 322 can include a model initializer circuit 324 and a model update circuit 325. The model initializer circuit 324 can be configured to initialize a model type and the model configuration for the partial predictor circuit K 320k. The model type can include one or a combination of two or more of a rule-based model, a decision tree model, a regression model, a neural network model, a random forest, a voting model, a fuzzy logic model, or a support vector machine model, among others. The model configuration can include detailed components and structure of a particular type of model. For example, for a decision tree model, its configuration can include nodes, paths, and tree levels that constitute the structure of a decision tree. Examples of the decision tree model and the ensemble of predictors each including a respective decision tree model are described below, such as with reference to FIG. 4.

The model initializer circuit 324 can be coupled to the instruction receiver circuit 250 and receive, among other programming commands, selection or confirmation of the selected model type and initial model configuration from an end-user via a user interface and programming device such as implemented in the external system 120. For example, the user interface and programming device can allow a user to program an initial DCM as a three-level decision tree model with specified nodes and branches. The model initializer circuit 324 can be alternatively coupled to a memory circuit that stores a plurality of candidate models with pre-determined model type or model configuration, and the model initializer circuit 324 can select from the memory a model for the partial predictor circuit K 320k. The selection can be achieved with interventions of an end-user such as via a user interface and device programming device. The selection can also be achieved automatically via a specified selection method. In an example, the model initializer circuit 322 can randomly select a model from the memory. In another example, the candidate models in the memory can be indexed by the target manifestation (e.g., cardiac or pulmonary events) or characteristics of the signal features, and the model initializer circuit 322 can select a model from the memory based on a specified manifestation of the worsening of HF, or the signal features used by the partial predictor circuit. For example, the model initializer circuit 322 can select a neural network model if the signal features are used to predict warm-and-wet or cold-and-dry profiles of the HF, select a decision tree model if the signal features are used to predict central or peripheral congestion, or select a support vector machine model if the signal features are used to predict cardiac or pulmonary events precipitating a worsening of HF.

The model update circuit 325 can adaptively update the DCM selected or generated by the model initializer circuit 324. For a selected model type, the model update can include changes of one or more elements of the model configuration when a specified condition is met, such as the performance of the partial predictor circuit K 320k meets a specified criterion. For example, when the model initializer circuit 324 initialize the DCM to a decision tree model, the model update circuit 325 can add or remove one or more of a tree node, a path, or a tree level that constitute the decision tree model.

The model update circuit 325 can adapt the DCM to the received physiologic signal, such as by updating the structure or complexity of the model using a feedback mechanism. The model update circuit 325 can evaluate the performance of the DCM using the physiologic signals such as sensed by the physiologic signal receiver circuit 210. The model update circuit 325 can also be communicatively connected to a memory circuit and received historical physiologic data collected from a patient or from a cohort of patients with similar manifestations of worsening of HF. The model update circuit 325 can calculate a performance measure of the DCM and update the DCM using the performance measure. The performance measure can include a comparison between the model output and an adjudication such as received from a health-care professional or an end-user. The adjudication can include an adjudicated classification of the precursor physiologic event, such as presence of absence of a cardiac or a pulmonary event, a central or a peripheral congestion, or a wet-and-warm or a cold-and-dry hemodynamic profile of the HF status. Alternatively or additionally, the adjudication can include a decision of the presence or absence of an HF decompensating event or worsening of HF status. The performance measure can take the form of accuracy rate, error rate, sensitivity, specificity, positive predictive value, or negative predictive value, among others. If the performance measure does not meet a specified criterion, the model update circuit 325 can update the model configuration, such as by adding or pruning a branch, adding or removing a node, or extending or reducing a level of a decision tree model. The model update circuit 325 can receive more physiologic data and keep the adaptation process until a desirable performance is achieved, such as the error rate falling below a specified threshold, or a performance convergence criterion being met (e.g., the difference in performance measure before and after a model update falling below a specified threshold). In an example, the adaptation of the DCM can be achieved by randomly feeding the physiologic data to the DCM.

The DCMs created for the various partial predictor circuits in the ensemble of predictors 320 can be of different types or have different configurations. For example, the ensemble of predictors 320 can include an ensemble based learning structure such as a random forest that comprises a plurality of decision tree models. Each decision tree model can be part of the respective partial predictor circuit. The configuration of each decision tree model can be independently initialized and updated. The model update circuit of each partial predictor can use distinctive portions of the physiologic data to update the respective decision tree model. For example, the partial predictor circuit A can use a first portion of the physiologic data to update a decision tree model for predicting a central congestion, and the partial predictor circuit B can use a second portion of the physiologic data to update a neural network model for predicting a peripheral congestion. The first and second data portions can be respectively selected from one or more physiologic signals. The portions of physiologic data used by various partial predictor circuits can be randomly selected. In some examples, the second data portion is non-identical to the first data portion.

In some examples, some or all of the DCM circuits used by the ensemble of partial predictors, such as the DCM circuit 322, can be implemented in a device or a system external to the ensemble of predictors 320, and the updated DCM can be communicated to the respective partial predictor circuit for use in computing respective partial risk index. For example, the DCM circuit 322, along with the DCM circuits of some or all the other partial predictor circuits, can be implemented in a DCM unit external to the such as disposed in the external system 120. The DCM unit can be communicatively coupled to the IMD in which the classifier-fusion based HF event prediction circuit 300 (excluding the DCM circuits that are in the DCM unit external to the IMD) can be implemented. The external DCM unit can be configured to initialize a DCM for each partial predictor circuit in the ensemble of predictors 320. The external DCM circuit can be communicatively coupled to the physiologic signal receiver circuit 210 or a memory circuit that stores historical physiologic data collected from the patient or from a cohort of patients with similar manifestations of worsening of HF, and use the physiologic data to adaptively update the DCM when a model performance measure meets a specified criterion. In an example, the external DCM circuit can initialize and update the DCM for each partial predictor independently.

The partial risk calculator circuit 323 can be configured to calculate a partial risk index using the one or more candidate signal features such as generated by the feature generator circuit 321 and the DCM such as created by the DCM circuit 322. The partial risk index characterizes a particular aspect of the patient's pathophysiology related to the worsening of HF. The partial risk calculator circuit 323 can include one or both of a categorical predictor circuit 326 or a probability estimator circuit 327. The categorical predictor circuit 326 can classify the partial risk index into one of two or more classes each being a categorization of patient being presented with or absent of a particular pathophysiologic manifestation, or a categorized severity of such a manifestation, such as “pulmonary edema”, “no pulmonary edema”, “mild pulmonary edema”, “high risk of arrhythmia”, “low risk of arrhythmia”, “central congestion”, “peripheral congestion”, “no congestion”, “wet-and-warm profile”, “cold-and-dry profile”, etc. The probability estimator circuit 327 can calculate a probability or continuous quantity indicating the likelihood of the patient later developing a physiologic event such as a pulmonary or a cardiac event, a central or a peripheral congestion event, or other events indicative of worsening of HF.

The partial risk indices from the various partial predictor circuits in the ensemble of predictors 320, either in the form of categorical decisions or continuous variables, can then be combined at the prediction fusion circuit 230, which can generate a composite risk indicator (CRI). Depending on the format (categorical or numerical) of the partial risk indices, the prediction fusion circuit 230 can compute the CRI using decision fusion or probability fusion. The prediction fusion circuit 230 can also be used to identify patients at elevated risk of developing a new or worsening of an existing disease, such as pulmonary edema, pulmonary condition exacerbation, asthma and pneumonia, myocardial infarction, dilated cardiomyopathy, ischemic cardiomyopathy, systolic HF, diastolic HF, valvular disease, renal disease, chronic obstructive pulmonary disease, peripheral vascular disease, cerebrovascular disease, hepatic disease, diabetes, asthma, anemia, depression, pulmonary hypertension, sleep disordered breathing, or hyperlipidemia, among others.

FIG. 4 illustrates an example of an ensemble of predictors 420 and the prediction fusion of the output from the ensemble of predictors. The ensemble of predictors 420 can be an embodiment of the ensemble of predictors 220 or 320. The ensemble of predictors 420, referred to as random forest, can include a plurality of partial predictors each including a decision tree model. An example of the random forest 420 as illustrated in FIG. 4 includes a total of R decision tree models, denoted by tree₁ 420A, tree₂ 420B, . . . , and tree_{R} 420R. Each decision tree model, such as exemplary decision tree 420A, can include multiple nodes 421, branches 422, and one or more levels branching from respective node. A node of the decision tree model can represent a specified physiologic signal feature, such as a temporal or amplitude measurement from an electrogram, representative thoracic impedance, a change in respiration rate or tidal volume, or an S3 heart sound measurement, among others. A branch extending from the node can represent the signal feature at the node meeting a specified criterion, such as exceeding or falling below a threshold value. The levels of the decision tree model can represent the amount of signal features used for generating the partial risk index, thereby representing the sophistication of the decision tree model.

The decision tree model 420A can receive as input one or more of multi-sensor or multi-modal physiologic signals X={x₁, x₂, . . . , x_{N}} where xᵢ represents a sensor signal or a signal feature calculated from a physiologic signal. Each decision tree model within the random forest 420 can be independently initialized and updated. For example, nodes, branches, or levels of one decision tree model can be different from the respective elements of another decision tree model within the same random forest 420. In some examples, the initial tree configuration can be randomly selected, such that nodes at different levels can be represented by randomly selected signal features, or the number of nodes used in each tree or the levels of the tree can be randomly selected. The decision tree model can be updated adaptively using physiologic signals such as from 310 or from a memory storing historical physiologic data collected from the patient or from a cohort of patients with similar manifestations of worsening of HF. The model output from the decision tree (such as a cardiac or a pulmonary event, a wet-and-warm or a cold-and-dry profile, or a central or a peripheral congestion) can be compared to an adjudicated manifestation or characterization such as provided by a health-care professional or an end-user. A performance measure of the computation model based on comparison between the adjudication and the model output can be determined. Examples of the performance measure can include accuracy rate, error rate, sensitivity, specificity, positive predictive value, or negative predictive value, among others. If the performance measure does not meet a specified criterion, the decision tree can be update by adding or pruning a branch, adding or removing a node, or extending or reducing a level. The model update process can continue until a desirable performance is achieved, such as the error rate falling below a specified threshold, or the performance convergence criterion being met. In an example, the adaptation of the decision tree can be achieved by randomly feeding the physiologic data to the decision tree models.

The output of the decision tree model, denoted as kᵢ, represents the partial risk index which can characterize a particular aspect of the patient's pathophysiology indicative or correlative of the worsening of HF. The partial risk index kᵢ can be in the form of categorical decision such as one of two or more classes each being a categorization of patient being presented with or absent of a particular pathophysiologic manifestation, or a categorized severity of such a manifestation, such as “pulmonary edema”, “no pulmonary edema”, “mild pulmonary edema”, “high risk of arrhythmia”, “low risk of arrhythmia”, “central congestion”, “peripheral congestion”, “no congestion”, “wet-and-warm profile”, “cold-and-dry profile”, etc. The partial risk index kᵢ can alternatively take the form of a continuous quantity such as a probability value indicating the likelihood of the patient later developing a physiologic event such as a pulmonary or a cardiac event, a central or a peripheral congestion event, or other events indicative of worsening of HF.

The partial risk indices from some or all of the decision tree models can be combined at 230 using a decision fusion or a probability fusion K=ƒ(k₁, k₂, . . . , k_{R}). The composite risk indicator K can be a categorical decision or a confidence level of patient developing a future HF event. The fusion function ƒ can include a decision fusion method including majority voting, X-out-of-Y voting, or weighted voting, among others. Alternatively, the fusion function ƒ can include a probability fusion method including a linear or a non-linear combination such as average or weighted summation, or a parametric or a non-parametric method such as a decision tree, a neural network, a Bayesian network, among other machine learning methods. The decision fusion K can be reported to an end-user or to be used by a medical system such as the IMD in making therapy decisions.

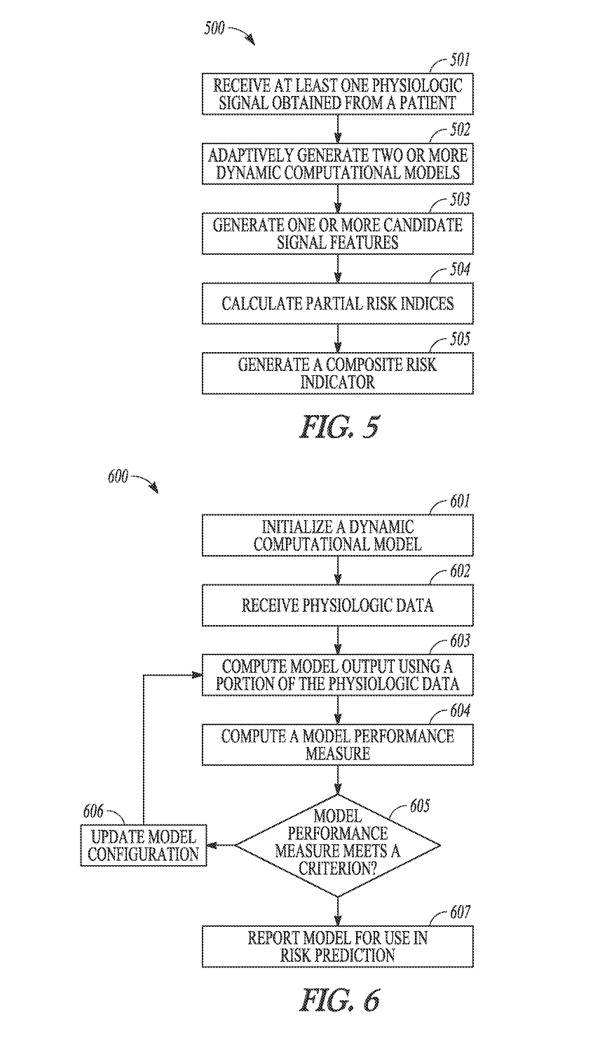

FIG. 5 illustrates an example of a method 500 for predicting a patient's risk of developing an event indicative of worsening of HF. The method 500 can be implemented and operate in an ambulatory medical device or in a remote patient management system. In an example, the method 500 can be performed by the classifier-fusion based HF event detection or risk assessment circuit 113 implemented in the IMD 110, or in the external device 120 which can be in communication with the IMD 110. In addition to predicting worsening of HF, the method 500 can be modified for use in identifying patients at elevated risk of developing a new or worsening of an existing disease, such as pulmonary edema, pulmonary condition exacerbation, asthma and pneumonia, myocardial infarction, dilated cardiomyopathy, ischemic cardiomyopathy, systolic HF, diastolic HF, valvular disease, renal disease, chronic obstructive pulmonary disease, peripheral vascular disease, cerebrovascular disease, hepatic disease, diabetes, asthma, anemia, depression, pulmonary hypertension, sleep disordered breathing, or hyperlipidemia, among others.

At 501, at least one physiologic signal obtained from a patient can be received. The physiologic signals can be indicative of worsening of HF status or other existing disease. The physiologic signals can be sensed using one or more physiologic sensors associated with the patient. Examples of such a physiological signal can include one or more electrograms sensed from the electrodes on one or more of the leads 108A-C or the can 112, electrocardiogram, heart rate, heart rate variability, intrathoracic impedance, intracardiac impedance, arterial pressure, pulmonary artery pressure, left atrial pressure, RV pressure, LV coronary pressure, coronary blood temperature, blood oxygen saturation, one or more heart sounds, physiologic response to activity, apnea hypopnea index, one or more respiration signals such as a respiration rate signal or a tidal volume signal. The physiologic signals can also include one or more of brain natriuretic peptide (BNP), blood panel, sodium and potassium levels and glucose level. The physiologic signals can be acquired from a patient and stored in a storage device such as an electronic medical record (EMR) system. The physiologic signals can be preprocessed or conditioned, including signal amplification, digitization, or filtering, among others.

At 502, two or more dynamic computational models (DCMs) can be generated. Each DCM can include one or a combination of various model types including a rule-based model, a decision tree model, a regression model, a neural network model, a random forest, a voting model, a fuzzy logic model, or a support vector machine model, among others. A DCM can be directed to a specified patient group that has certain types of comorbidities or certain triggering events. A DCM can include several elements that constitute the configuration of the model. For example, a decision tree model can include such elements as nodes, paths, and tree levels. With a determined model type and model structure, a DCM is capable of receiving input signals or signal features, processing the input according to a set of executable instructions, and generating a model output.

Types and configurations of the two or more DCMs at 502 can be independently initialized. In an example, the initial DCM can be randomly selected from a plurality of candidate computational models stored in a memory. Each DCM can then be independently updated such as using a feedback mechanism. Adaptation of a DCM can include update of the structure or complexity of the model when the model output meets a specified criterion. Examples of adaptation of the DCM are described below, such as with reference to FIG. 6.

At 503, one or more candidate signal features can be generated from the received physiological signals. Examples of signal features can include: signal mean, median, or other central tendency measures; a histogram of the signal intensity; one or more signal trends over time; one or more signal morphological descriptors; or signal power spectral density at a specified frequency range. The signals features can include components corresponding to physiologic activities. For example, the electrocardiogram or electrogram features can include P wave, R wave, T wave, QRS complex, or other components representing depolarization, hyperpolarization, repolarization, or other electrophysiological properties of the myocardium. The heart sound features can include timing, amplitude, or morphologic characteristics of one or more of S1, S2, S3, or S4 heart sounds. The thoracic impedance features can include maximum, minimum, mean, variance, rate of change, or other statistical or morphological features. The respiration signal features can include respiration rate, respiration depth, tidal volume, or other descriptors.

Different physiologic signals, or different signals features calculated from the same or different physiologic signals, can be used as input to the two or more DCMs. By using different physiologic signals or different signal features, various DCMs can characterize different aspects of the patient's pathophysiology indicative or correlative of the worsening of HF. For example, a first DCM can use signal features generated from a thoracic impedance signal, while a second DCM can use signal features generated from an acoustic or vibrational heart sound signal.

At 504, a partial risk index can be computed using respective DCM and respective candidate signal features. In an example where at least first and second DCMs are used in predicting patient's risk of developing a future event of worsening of HF, the partial risk indices generated from the first and second DCMs can represent the patient's risk of developing different physiologic events or pathophysiologic manifestations such as precipitating worsening of HF. Examples of such pathophysiologic manifestation can include a cardiac event (e.g., cardiac arrhythmia), a pulmonary event (e.g., a pulmonary edema), a renal event (e.g., glomerular filtration rate), a hypertension, a diabetic condition, a central congestion, a peripheral congestion, a warm-and-wet or a cold-and-dry profile suggestive of patient's hemodynamic presentation of the HF status. The partial risk index can be in a form of a categorical decision such as one of two or more classes each being a characterization of patient being presented with or absent of a particular pathophysiologic manifestation, or a categorized severity of such a manifestation, such as “pulmonary edema”, “no pulmonary edema”, “mild pulmonary edema”, “high risk of arrhythmia”, “low risk of arrhythmia”, “central congestion”, “peripheral congestion”, “no congestion”, “wet-and-warm profile”, “cold-and-dry profile”, etc. The partial risk index can alternatively take the form of a continuous quality such as a probability value indicating the likelihood of the patient later developing a physiologic event such as a pulmonary or a cardiac event, a central or a peripheral congestion event, or other events indicative of worsening of HF.

At 505, the partial risk indices from the various DCMs can be combined to generate a composite risk indicator (CRI) indicating a likelihood of the patient developing a future event of worsening of HF. The CRI can be a classification decision taken from two or more classes indicating the likelihood of an impending event of worsening of HF. For example, when the partial risk indices calculated at 504 take the form of categorical decisions, the CRI can be calculated using decision fusion of the categorical partial risk indices. Examples of the decision fusion method can include majority voting, X-out-of-Y voting, or weighted voting, among others. The CRI can also be a continuous quality such as a probability value indicating a likelihood of occurrence of an impending event of worsening of HF. For example, when the partial risk indices calculated at 504 take the form of probability values indicating the confidence level of a prediction of an impending pathophysiologic manifestation, the CRI can be computed using probability fusion of the partial risk indices. Examples of the probability fusion method can include linear or a non-linear combination such as average or weighted summation, or parametric or non-parametric methods such as a decision tree, a neural networks, a Bayesian network, among other machine learning methods.

FIG. 6 illustrates an example of a method 600 for generating a dynamic computational model (DCM) used for predicting a precursor physiologic event indicative or correlative of impending worsening of HF. The method 600 can be an embodiment of the method 500 for adaptively generating the computational models. The method 600 can be performed by the DCM circuit 322 in any partial predictor circuit within the ensemble of predictors 320. The method 600 can alternatively be performed by a dynamic computational model unit external to an ambulatory medical device such as the IMD 110.

At 601, a DCM can be initialized. One or both of the model type and the initial model configuration can be programmed by an end-user, or selected from a plurality of pre-determined candidate models stored in a memory. The selection can be achieved automatically via a specified selection method. In an example, the type or the configuration of the initial DCM can be randomly selected such as through a random selection process from a plurality of candidate computational models stored in a memory, each model having a specified model type and model configuration. In another example, the initial DCM can be selected based on pathophysiologic manifestation of worsening of HF, including a cardiac event (e.g., a cardiac arrhythmia), a pulmonary event (e.g., a pulmonary edema), a central congestion, a peripheral congestion, a warm-and-wet profile or a cold-and-dry profile of suggestive of patient's hemodynamic presentation of the HF status. For example, a neural network model can be selected if the computational model is to predict warm-and-wet or cold-and-dry profiles of the HF, a decision tree model can be selected if the computational model is to predict central or peripheral congestion, or a support vector machine model can be selected if the computational model is to predict cardiac or pulmonary events precipitating a worsening of HF.

At 602, physiologic data can be received for adaptively updating the DCM. The physiologic data can include physiologic signals sensed by ambulatory physiologic sensors associated with the patient, or historical physiologic data collected from the patient or from a cohort of patients with similar manifestations of worsening of HF. At 603, a portion of the received physiologic data can be selected from the received physiologic data and fed into the DCM, and model output can be computed. The model output can be a categorical decision or classification of a precursor physiologic event indicative or correlative of worsening of HF, including a cardiac or a pulmonary event, a central or a peripheral congestion, or a wet-and-warm or a cold-and-dry hemodynamic profile of the HF status, among others. In some examples, the portions of physiologic data used by the DCM can be randomly selected. When two or more DCMs are used, different portions of the physiologic data can be used to compute the model output.

At 604, a performance measure of the DCM can be calculated. The performance measure can include a comparison between the model output and an adjudication such as received from a health-care professional or an end-user. The adjudication can include an adjudicated classification of the precursor physiologic event, such as presence of absence of a cardiac or a pulmonary event, a central or a peripheral congestion, or a wet-and-warm or a cold-and-dry hemodynamic profile of the HF status. Alternatively or additionally, the adjudication can include a decision of the presence or absence of an HF decompensating event or worsening of HF status. The performance measure can take the form of accuracy rate, error rate, sensitivity, specificity, positive predictive value, or negative predictive value. The model performance measure can also include measure of convergence, including a change, a rate of change, or other higher order differences between mode performances during the model update process.

At 605, a decision is made as to whether the model performance measure meets a specified criterion for continuing or terminating the model update. If the performance criterion is met at 605, such as the error rate falls below a specified threshold, then the DCM is deemed satisfactory, and a report can be generated at 607 to notify the end-user about the type and configuration of the DCM. The DCM can then be used in predicting an aspect or a pathophysiologic manifestation of an impending worsening of HF. However, if the performance criterion is not met at 605 such as the error rate is above the specified threshold, the DCM can be updated at 606 such as by altering the configuration of the computation model. In an example of a decision tree model, the model can be updated by adding or pruning a branch, adding or removing a node, or extending or reducing a level of the decision tree model. A randomly selected portion of the physiologic data can then be used to evaluate the performance of the updated DCM at 603.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A system, comprising:
a physiologic signal receiver circuit configured to receive at least one physiologic signal obtained from a patient;
two or more partial predictor circuits, each including:
a feature generator circuit configured to generate one or more candidate signal features from the at least one physiologic signal;
a dynamic computational model circuit configured to adaptively generate a dynamic computational model; and
a partial risk calculator circuit configured to calculate a partial risk index using the one or more candidate signal features and the dynamic computational model, the partial risk index indicating a likelihood of the patient developing a precursor physiologic event indicative or correlative of a future target physiologic event; and
a prediction fusion circuit coupled to the two or more partial predictor circuits, the prediction fusion circuit configured to generate a composite risk indicator using the partial risk indices produced by the two or more partial predictor circuits, the composite risk indicator indicative of a likelihood of the patient developing the future target physiologic event.

2. The system of claim 1, wherein the two or more partial predictor circuits differ from each other by at least one of the one or more candidate signal features or the dynamic computational model.

3. The system of claim 1, wherein the physiologic signal receiver circuit is configured to receive one or more physiologic signals including a thoracic impedance signal, a heart sound (HS) signal, respiration signal, a posture signal, an activity signal, a heart rate signal, or a physiologic response to activity (PRA) signal.

4. The system of claim 1, wherein the two or more partial predictor circuits include first and second partial predictor circuits, the first partial predictor circuit including a first partial risk calculator circuit configured to calculate a first partial risk index indicating a likelihood of the patient developing a first type of precursor physiologic event, the second partial predictor circuit including a second partial risk calculator circuit configured to calculate a second partial risk index indicating likelihood of the patient developing a second type of precursor physiologic event different from the first type of precursor physiologic event.

5. The system of claim 4, wherein the first partial risk calculator circuit is configured to calculate the first partial risk index indicating a likelihood of the patient developing a pulmonary event, and the second partial risk calculator circuit is configured to calculate the second partial risk index indicating a likelihood of the patient developing a cardiac event.

6. The system of claim 4, wherein the first partial risk calculator circuit is configured to calculate the first partial risk index indicating a likelihood of the patient developing a peripheral congestion, and the second partial risk calculator circuit is configured to calculate the second partial risk index indicating a likelihood of the patient developing a central congestion.

7. The system of claim 1, wherein the two or more partial predictor circuits include first and second partial predictor circuits, the first partial predictor circuit including a first dynamic computational model circuit configured to adaptively generate a first dynamic computation model using a first data portion, the second partial predictor circuit including a second dynamic computational model circuit configured to adaptively generate a second dynamic computation model using a second data portion, the first and second data portions respectively selected from one or more physiologic signals.

8. The system of claim 7, wherein the physiologic signal receiver circuit is configured to receive patient historical physiologic data, and wherein the first and second partial predictor circuits are configured to select the respective first and second data portions from the patient historical physiologic data.

9. The system of claim 7, wherein the first data portion is non-identical to the second data portion.

10. The system of claim 1, wherein the dynamic computational model circuit is configured to adaptively generate the dynamic computational model including one or a combination of two or more of a rule-based model, a decision tree, a regression model, a neural network model, a random forest, a voting model, a fuzzy logic model, or a support vector machine model.

11. The system of claim 1, wherein the dynamic computational model circuit is configured to initialize the dynamic computational model to a randomly-selected structure.

12. The system of claim 1, wherein:
the two or more partial predictor circuits each is configured to calculate a partial risk index including a categorical decision indicating occurrence of the physiologic event; and
the prediction fusion circuit is configured to generate the composite risk indicator using voting among the categorical decisions.

13. The system of claim 1, wherein:
the two or more partial predictor circuits each is configured to calculate a partial risk index including a probability value indicating a likelihood of the physiologic event; and
the prediction fusion circuit is configured to generate the composite risk indicator using a linear or a non-linear combination of the probability values.

14. A system, comprising:
a dynamic computational model unit, including:
a memory circuit configured to receive and store physiologic data; and
a model update circuit adaptively generate two or more dynamic computational models using the stored physiologic data; and
an ambulatory medical device communicatively coupled to the dynamic computational model unit, the ambulatory medical device including:
a receiver circuit configured to receive from the dynamic computational model unit the two or more dynamic computational models;
a physiologic signal receiver circuit configured to receive at least one physiologic signal obtained from a patient;
two or more partial predictor circuits configured to generate one or more candidate signal features from the at least one physiologic signal, and to calculate a partial risk index using the one or more candidate signal features and the two or more dynamic computational models, the partial risk index indicating a likelihood of the patient developing a precursor physiologic event indicative or correlative of a future target physiologic event; and
a prediction fusion circuit coupled to the two or more partial predictor circuits, the prediction fusion circuit configured to generate a composite risk indicator using the partial risk indices produced by two or more partial predictor circuits, the composite risk indicator indicative of a likelihood of the patient developing the future target physiologic event.

15. The system of claim 14, wherein the dynamic computational model unit is configured to adaptively generate the two or more dynamic computational models including one or a combination of two or more of a rule-based model, a decision tree, a regression model, a neural network model, a random forest, a voting model, a fuzzy logic model, or a support vector machine model.

16. The system of claim 14, wherein the two or more partial predictor circuits include a first partial predictor circuit and a second partial predictor circuit different from the first partial predictor circuit by at least one of the one or more candidate signal features or the dynamic computational model.

17. A method, comprising:
adaptively generating at least first and second dynamic computational models;
receiving at least one physiologic signal obtained from a patient;
generating one or more candidate signal features using the at least one physiologic signal;
calculating a first partial risk index using first signal features and the first dynamic computational model and calculating a second partial risk index using second signal features and the second dynamic computational model, the first and second signal features respectively selected from the one or more candidate signal features, the first partial risk index indicating a likelihood of the patient developing a first precursor physiologic event, the second partial risk index indicating a likelihood of the patient developing a second precursor physiologic event, the first and second precursor physiologic events indicative or correlative of a future target physiologic event;
generating a composite risk indicator using one or both of the first and second partial risk indices, the composite risk indicator indicative of a likelihood of the patient developing the future target physiologic event.

18. The method of claim 17, wherein adaptively generating the at least first and second dynamic computational models includes generating the first dynamic computational model having different type or different structure than the second dynamic computational model, the types of the models including a rule-based model, a decision tree, a regression model, a neural network model, a random forest, a voting model, a fuzzy logic model, or a support vector machine model.

19. The method of claim 17, wherein calculating the first partial risk index includes calculating a risk index indicating a likelihood of the patient developing a pulmonary event, and wherein calculating the second partial risk index includes calculating a risk index indicating a likelihood of the patient developing a cardiac event.

20. The method of claim 17, wherein generating the composite risk indicator includes taking a linear or nonlinear combination of the first and second partial risk indices.
